(19) United States
(12) Patent Application Publication
(10) Pub. No.: US 2026/0344806 A1
(43) Pub. Date: Nov. 5, 2026

(54) PUMPING BRA

(71) Applicant: Alisa VOLK, Portland, OR (US)

(72) Inventor: Alisa VOLK, Portland, OR (US)

(21) Appl. No.: 19/643,046

(22) Filed: Apr. 9, 2026

Related U.S. Application Data
(60) Provisional application No. 63/786,049, filed on Apr. 9, 2025.

(51) Int. Cl. A41C 3/04; A41C 3/00; A61F 7/00; A61M 1/06

(52) U.S. Cl. CPC A41C 3/04; A41C 3/0035; A61F 7/007; A61M 1/067; A61F 2007/0021; A61F 2007/0071; A61F 2007/008; A61M 2209/088

(57) ABSTRACT

Pumping bras that facilitate breast milk expression by applying gentle warming to breast tissue, enhancing comfort as well as breast milk expression, and easing the symptoms of clogged ducts or mastitis. The pumping bras include a pair of front panels, each front panel having a central front panel opening that is covered by a layered overlapping and interlocking central front panel opening cover, an inner compartment formed in each front panel, at least two heating units that can be inserted into the inner compartments, and a battery pack capable of energizing the heating units.

### TECHNICAL FIELD

The present disclosure relates generally to brassieres, or bras, and more particularly to a bra that facilitates the pumping of breast milk, including hands-free pumping, without removing the bra.

### BACKGROUND

Breast milk offers several advantages over manufactured formula as a food source for infants, and so it is beneficial to assist caregivers to feed infants with breast milk for as long as they may wish. For a variety of reasons, many caregivers may choose to use a breast pump to express breast milk, and by removing as many impediments to breast milk expression as possible, the duration of breast milk feeding may be extended. Furthermore, by reducing complications associated with breast milk expression, and by increasing comfort during expression, the likelihood that breast milk feeding can be continued as long as desired is increased.

Selected aspects of breast milk expression that may prove challenging include a slowed or reduced milk letdown, pain and discomfort associated with clogged milk ducts, or suffering from mastitis.

What is needed is a pumping bra that can facilitate breast milk expression by being comfortable yet supportive, making the use of a breast pump apparatus less complicated and awkward, and by helping address some of the conditions that can interfere with breast milk expression.

### SUMMARY

The present disclosure is directed to pumping bras that facilitate breast milk expression while simultaneously applying gentle warming to breast tissue, enhancing comfort as well as breast milk expression, and easing the symptoms of clogged ducts or mastitis.

In one example, the present disclosure is directed to a pumping bra, where the pumping bra includes a pair of front panels, each front panel including a support zone that is soft and unlined and configured to support one of a wearer's breasts, and each front panel having an inner fabric layer and an outer fabric layer. Each support zone defines a central front panel opening that is positioned to encircle a nipple of one of the wearer's breasts, each central front panel opening being substantially circular and having a circumference, and each central front panel opening being covered by a layered overlapping and interlocking central front panel opening cover. Each central front panel opening cover includes an outer pair of flap members and an inner pair of flap members, the outer pair of flap members having a first outer fabric flap member and a second outer fabric flap member that extend from opposing sides of the central front panel opening so that an inner edge of the first outer fabric flap member overlaps an inner edge of the second outer fabric flap member along a first chord of the substantially circular central front panel opening; the inner pair of flap members having a first inner fabric flap member and a second inner fabric flap member that extend from opposing sides of the central front panel opening so that an inner edge of the first inner fabric flap member overlaps an inner edge of the second inner fabric flap member along a second chord of the central front panel opening, provided that the first chord and the second chord are not parallel, so that each central front panel opening cover is configured such that by simultaneously separating the outer pair of flap members and separating the inner pair of flap members an access is provided to the nipple encircled by that central front panel opening.

A compartment is formed between the inner fabric layer and the outer fabric layer of each front panel support zone, the compartment having a compartment perimeter where the inner fabric layer and the outer fabric layer are joined, so that the resulting compartment extends at least partially around the circumference of the corresponding central front panel opening, and each front panel defines an opening to the corresponding compartment that provides access to an interior of the compartment of that front panel.

The pumping bra includes at least two heating units, where each heating unit is configured to be inserted into the compartment of one of the front panels via the corresponding compartment opening and positioned within the compartment. Each heating unit can be electrically coupled to a battery pack capable of energizing the heating unit while it is inserted into the compartment and warming the wearer's body where that heating unit is positioned.

The pumping bra includes a back portion that rests against the wearer's back and is coupled to one of the pair of front panels on each side of the back portion. The back portion further includes at least one external pocket to receive the battery pack while the battery pack is coupled to one or more heating units.

The pumping bra includes a front fastener, disposed between the pair of front panels to fasten the pumping bra on the wearer, and a pair of shoulder straps extending from the pair of front panels to the back portion, the shoulder straps having a racerback configuration to support the pumping bra on the wearer.

Selected embodiments of the nursing bra and aspects thereof are described and illustrated in the present application. Such descriptions and illustrations are meant to be exemplary and illustrative, and should not be considered to be limiting in scope.

### BRIEF DESCRIPTION OF THE DRAWINGS

FIG. 1 is a front view of an exemplary pumping bra according to the present disclosure.

FIG. 2 is a rear view of the pumping bra of FIG. 1.

FIG. 3 depicts an exemplary power supply and heating elements for an exemplary pumping bra according to the present disclosure.

FIG. 4A shows the front of the left panel of the exemplary pumping bra; FIG. 4B shows the rear of the left panel of the exemplary pumping bra.

FIG. 5 semi-schematically depicts the insertion of a breastpump flange into an exemplary pumping bra according to the present disclosure.

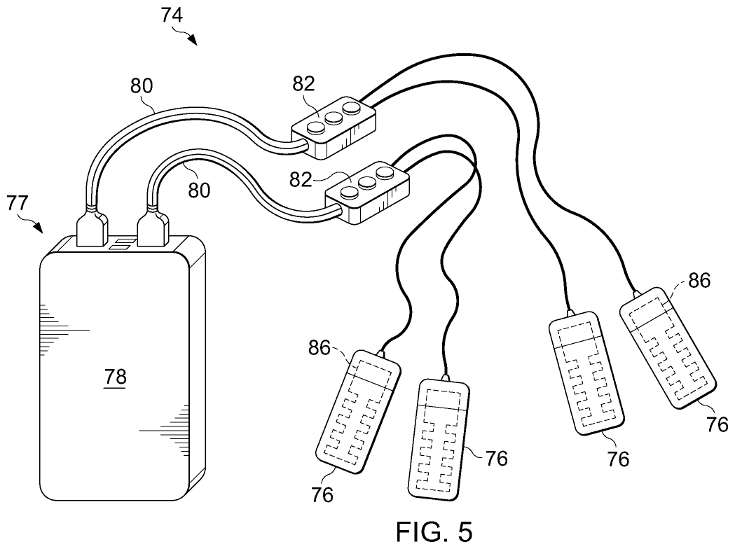

### DETAILED DESCRIPTION OF PREFERRED EMBODIMENTS

The following is a detailed description of selected exemplary embodiments to illustrate the principles of the disclosed pumping bra, but the invention is not limited to any embodiment. Rather, the scope of the invention may encompass numerous alternatives, modifications and equivalents without departing from the scope of the claims.

The present disclosure is directed to pumping bras that facilitate breast milk expression by applying heat to breast tissue. By providing additional warmth during expression let-down is encouraged and milk flow is increased, clogged milk ducts may be cleared, the chance of clogging is reduced, and the comfort of the wearer is enhanced.

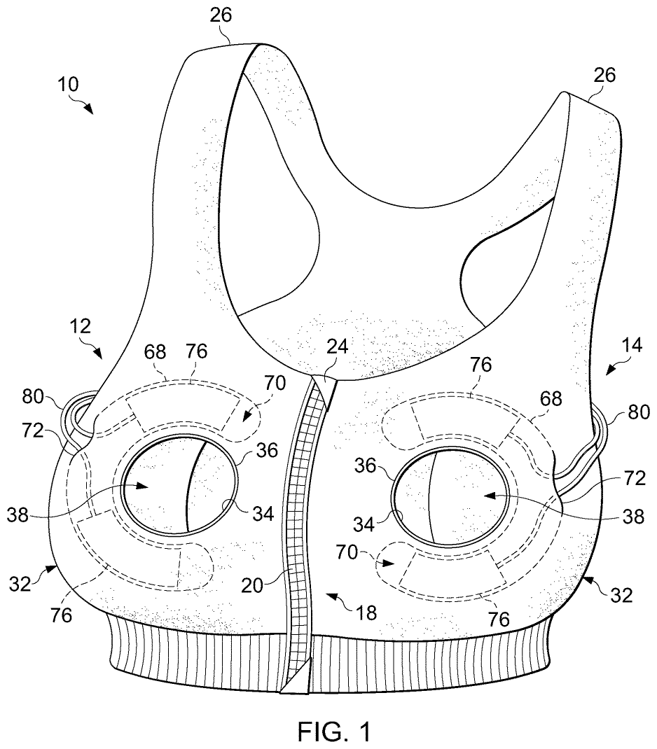

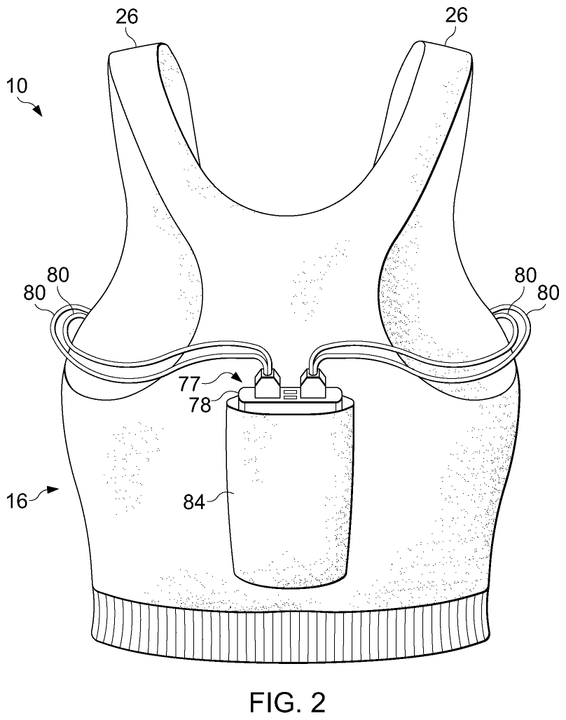

FIGS. 1 and 2 depict an exemplary pumping bra 10 according to the present disclosure. As shown, pumping bra 10 includes a pair of front panels 12 and 14, and a back portion 16 that is configured to rest against the wearer's back. Back portion 16 is coupled to both front panels 12 and 14, at opposing sides of the back portion.

Front panels 12 and 14 are coupled together by a front fastener 18. Front fastener 18 is disposed between the front panels 12 and 14, and serves to fasten the pumping bra 10 on the wearer. Any type of fastener that is generally used for bras may be a suitable front fastener for the purposes of the present disclosure. Front fastener 18 is typically selected so as to enhance the support provided by pumping bra 10, and to provide durability during use and laundering. Preferably, front fastener 18 may include a vertically-oriented zipper 20 that connects front panels 12 and 14. When front fastener 18 includes a zipper 20, the zipper may be of high quality, and include a self-locking function to prevent the zipper slider from working its way downward.

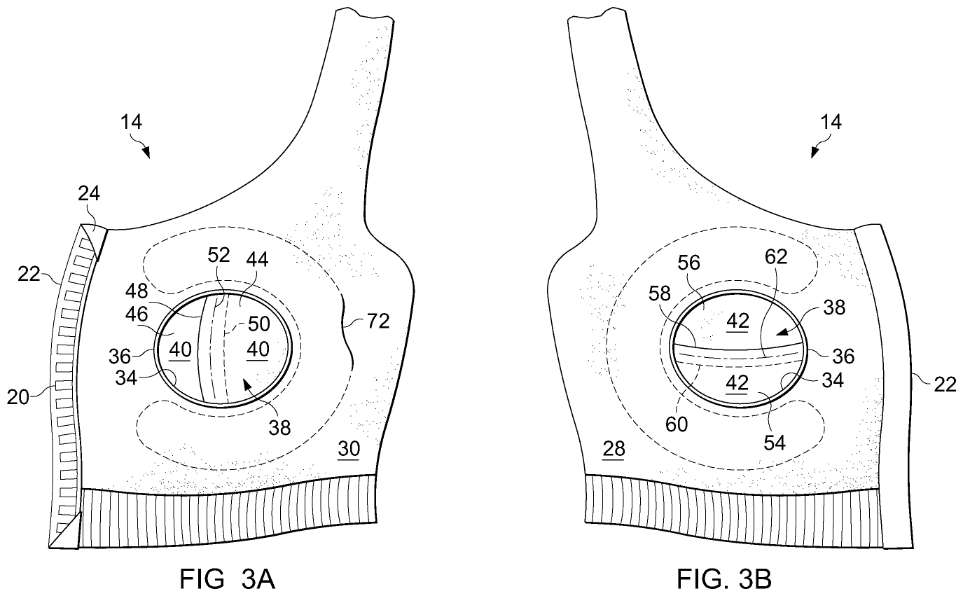

As shown in FIGS. 3A and 3B, a protective fabric fastener flap 22 preferably extends from an inner surface of one of front panels 12 and 14 adjacent to front fastener 18 in order to prevent contact between the front fastener and the wearer's skin and avoid possible chafing. The presence of fastener flap 22 may be particularly advantageous where front fastener 18 includes zipper 22. In addition to fastener flap 22, the front panel adjacent to the portion of zipper 22 may additionally include a ‘garage’ 24, or a fabric enclosure at a terminal end of zipper 22 configured for a zipper slide to be inserted into, to provide additional comfort and to help prevent unwanted unfastening of pumping bra 10.

A pair of shoulder straps 26 extend from the front panels 12 and 14 of pumping bra 10 to back portion 16 in such a way as to suspend the pumping bra 10 from the wearer's shoulders and provide additional support. In particular, and as shown in FIGS. 1 and 2, shoulder straps 26 may be designed to embody a “racerback” configuration. A racerback style of bra includes shoulder straps that are coupled, or connected, to one another above the back portion of the bra. The use of a racerback configuration of shoulder straps permits pumping bra 10 to provide enhanced support for the wearer. In particular, the racerback configuration can reduce the weight supported by the bra at the shoulders, and distributes the supported weight toward the center of the wearer's back, helping to reduce shoulder strain and pain. The connection between the shoulder straps that is typical for a racerback configuration can include a t-shaped strap that connects the left and right straps above the back portion, or, as shown in FIG. 2, rather than having each shoulder strap connect independently with the back portion, the right and left shoulder straps may merge above the back portion and form a single connection to the back portion of the pumping bra.

Pumping bra 10 can be a compression bra. That is, pumping bra 10 can be manufactured with compression fabric, in order to provide comfort and support to the wearer of the pumping bra. Compression fabrics typically incorporate an elastic polymer in the fabric in order to confer the desired resilience, such as those polyurethane polymers known as SPANDEX, LYCRA, or elastane. Suitable fabrics may include cotton/SPANDEX blends, modal/SPANDEX blends, or nylon/SPANDEX blends, among many others.

In particular, front panels 12 and 14 can include a compression fabric and can be configured to provide comfort and support to the wearer of the pumping bra. Each of front panels 12 and 14 include an inner fabric layer 28 and an outer fabric layer 30 that form a support zone 32 that is soft and unlined, but provides lift and support via the elasticity of the compression fabric used without requiring distinct cups, as are used for encapsulation-type bras.

The support zone 32 of each front panel 12 and 14 includes and defines a central front panel opening 34 in a central area of each support zone. Each central front panel opening 34 is typically substantially circular in shape, and has a substantially circular circumference 36. Each central front panel opening 34 is disposed and positioned within its front panel so that when pumping bra 10 is being worn, the central front panel opening 34 encircles a nipple of the wearer's breast. That is, each central front panel opening 34 is placed and configured so as to provide access to the wearer's nipple in order to enable the expression and collection of breast milk.

Each central front panel opening 34 is covered by a central front panel opening cover 38. Central front panel opening cover 38 provides temporary access to the wearer's nipple for the expression and collection of breast milk, via a layered, overlapping, and interlocking construction. More specifically, each central front panel opening cover 38 includes an outer pair of flap members 40 and an inner pair of flap members 42. Outer pair of flap members 40 are formed by a first outer fabric flap member 44 and a second outer fabric flap member 46, where the first and second out flap members extend from opposing sides of the central panel opening 30, so that an inner edge 48 of the first outer fabric flap member overlaps an inner edge 50 of the second outer fabric flap member along a first chord 52 of the substantially circular central front panel opening. For the purposes of this disclosure, a chord of a circle is defined as a substantially straight segment whose endpoints both lie on the circumference of the circle.

For example, FIG. 3A shows the front surface of front panel 14, which includes the central front panel opening 34. Central front panel opening 34 is covered by central front panel opening cover 38 formed by an outer and an inner pair of flap members. Outer pair of flap members 40 includes the first outer fabric flap member 44 and second outer fabric flap member 46, where first outer fabric flap member 44 and second outer fabric flap member 46 are configured so that the inner edge 48 of first outer fabric flap member 44 overlaps the inner edge 50 of second outer fabric flap member 46. The area of overlap of flap members 44 and 46 form a substantially straight first chord 52 of the central front panel opening 34.

FIG. 3B shows the rear surface of front panel 14, and so the inner pair of flap members 42 is visible in the drawing. Inner pair of flap members 42 includes the first inner fabric flap member 54 and the second inner fabric flap member 56, where first inner fabric flap member 54 and second inner fabric flap member 56 are configured so that the inner edge 58 of first inner fabric flap member 54 overlaps the inner edge 60 of second inner fabric flap member 56. The area of overlap of flap members 54 and 56 form a substantially straight second chord 62 of the central front panel opening 34.

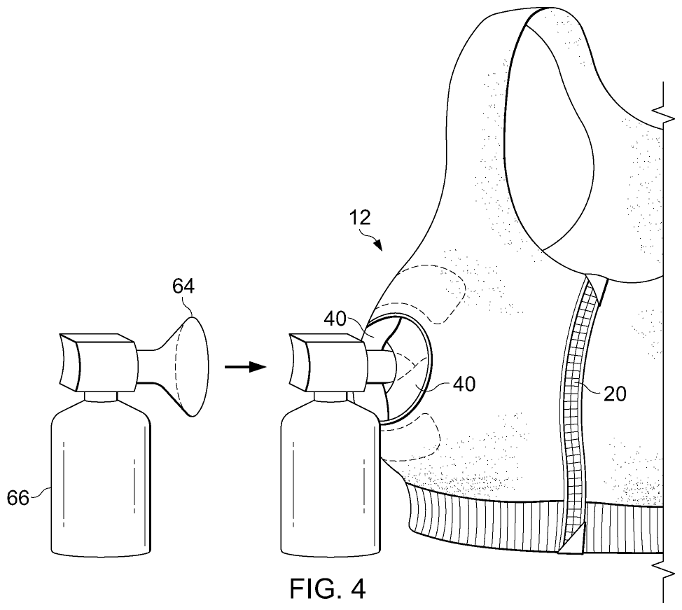

Access to the wearer is provided by manually and simultaneously separating the outer pair of flap members 40 and the inner pair of flap members 42. Once access is provided, breast milk may be expressed and collected, such as by a breast pump. FIG. 4 shows the an exemplary insertion of a flange 64 of a breast pump collection bottle 66 through the central front panel opening 34 by separating outer pair of flap members 40 and the inner pair of flap members 42. Flange 64 may be securely retained by the elasticity of the fabric forming the inner and outer flap members. This security may be enhanced by ensuring that the first chord 52 defined by the outer flap members is not parallel to the second chord 58 of the inner flap members. More preferably, first chord 52 is at an angle greater than about 45 degrees from second chord 58, and yet more preferably first chord 52 is disposed at substantially a right angle (i.e. perpendicular) to second chord 58. Due to this construction it may be possible to retain flange 64 within pumping bra 10 without the need of holding and supporting flange 64. That is, the wearer may then pump substantially hands-free.

For each front panel 12 and 14, inner fabric layer 28 and outer fabric layer 30 create an intervening space between the fabric layers. By sealing inner fabric layer 28 and outer fabric layer 30 to one another along a compartment perimeter 68, an inner compartment 66 within the front panel may be formed. Inner fabric layer 28 and outer fabric layer 30 may be sealed by any suitable means, including stitching, adhesive, heat-sealing or other method, although stitching along compartment perimeter 68 may be the most effective and efficient method of forming inner compartment 70.

Compartment perimeter 68 may be configured so that the resulting compartment 70 extends at least partially around central front panel opening 34. That is, so that compartment 70 is adjacent to central front panel opening 34, and extends along at least a portion of central front panel opening circumference 36. Typically, inner compartment 70 will extend around at least 50% of central front panel opening circumference 36. More preferably, inner compartment 70 extends around more than 50% of central front panel opening circumference 36. Each front panel 12 and 14 defines an opening 72 in outer fabric layer 24 that provides access to an interior of inner compartment 70 within that front panel. Any opening style may be used to provide access to the interior of inner compartment 70, but a simple slit may be a particularly advantageous configuration for opening 72.

Warming of the wearer's breast tissue may be accomplished by a heating system 74, which may be incorporated into pumping bra 10. heating system 74 may include at least one heating unit 76 that is configured to be inserted into the inner compartment 70 of a front panel. Insertion of heating unit 76 is accomplished via opening 72 to inner compartment 70. Heating unit 76 is typically configured to be easily inserted into inner compartment 70, and each heating unit 76 may be sized appropriately that its position within inner compartment 70 can be adjusted as desired, thereby localizing the source of heat where the wearer wishes. In some cases, more than one heating unit 76 can be inserted into inner compartment 70 in order to provide greater flexibility in placement of heat sources, and greater heating capacity. For example, as shown in FIG. 1, each inner compartment 70 may contains two individual heating units 76.

Heating system 74 may further include a power source capable of energizing the heating units 76 while they are disposed within inner compartment 70. The portability of pumping bra 10 may be enhanced by using a power source that is a battery pack 78. A variety of battery packs are commercially available suitable for use as power sources/external batteries for cellular phones and other portable electronics, and such battery packs may be used with the heating units 76 of pumping bra 10.

Each heating unit 76 may be electrically coupled to battery pack 78 via any suitable electrical connection 80, such as a power cable. In order to employ readily available technology, electrical connection 80 and battery pack 78 may be configured to be connected via a cable employing a powered USB connection, as a variety of standard formats exist for powered USB connectors. In one aspect, as shown in FIG. 4, each heating unit 76 is permanently attached to an electrical connection 80 that is also configured to be reversibly connected to battery pack 78.

Optionally, heating system 74 may further include a heating control 82 to regulate the heat output of heating units 76. Typically, heat is produced by heating units 76 via conventional resistive heating, and so the heat output of heating units 76 may be controlled by controlling the voltage delivered to the heating units. Any controller capable of controlling the heat output of heating units 76 is a suitable controller for the purposes of this disclosure. However, a heating control 82 that is disposed on and/or as a part of electrical connection 80 (such as by being interposed in the cabling) may be particularly advantageous. Preferably, each heating control 82 is configured to controllably provide multiple distinct heat outputs by the associated heating unit 76. More preferably, each heating control 82 may be configured to controllably provide 3-5 distinct heat outputs by the associated heating unit 76

A pocket 84 may be formed on back portion 16 of pumping bra 10 that is sized and configured to accept the battery pack 78, as shown in FIG. 2. Electrical connections 76 may extend from battery pack 78 in pocket 84 to heating units 76 where they are disposed within inner compartment 70 as shown in FIG. 1.

Heating units 76 may be any suitable resistive heating unit, provided that the resistive heating units are physically compatible with insertion into inner compartment 70 via opening 72. That is, a preferred heating unit 76 should be both substantially thin or flat, and at least somewhat flexible, so that each heating unit 72 can conform to the shape of the wearer's breast as desired. In particular, heating pads that employ carbon fiber elements as heating elements may be particularly advantage for use as heating unit 76. For example, as shown in FIG. 4, heating units 76 incorporate a heating element 82 that is a carbon fiber heating element. Suitable carbon fiber heating elements and heating pads are widely commercially available.

Although the pumping bra of the present disclosure has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

### REFERENCE NUMBERS

Ref. No.
10 Pumping bra
12 Front panel
14 Front panel
16 Back portion
18 Front fastener
20 Zipper
22 Fastener Flap
24 Garage
26 Shoulder strap
28 Inner fabric layer
30 Outer fabric layer
32 Support zone
34 Central front panel opening
36 Central front panel opening circumference
38 Central front panel opening cover
40 Outer pair flap members
42 Inner pair flap members
44 First outer fabric flap member
46 Second outer fabric flap member
48 Inner edge first outer flap member
50 Inner edge second outer flap member
52 First chord of the central front panel opening
54 First inner fabric flap member
56 Second inner fabric flap member
58 Inner edge first inner flap member
60 Inner edge second inner flap member
62 Second chord of the central front panel opening
64 Flange of breast pump
66 Breast pump collection bottle
68 Compartment perimeter
70 Inner compartment
72 Opening to inner compartment
74 Heating system
76 Heating unit
78 Battery pack
80 Electrical connection
82 Heating control
84 Pocket
86 Heating element

1. A pumping bra, comprising:
a pair of front panels, each front panel including a support zone that is soft and unlined and configured to support one of a wearer's breasts, and each front panel having an inner fabric layer and an outer fabric layer;
wherein each support zone defines a substantially circular central front panel opening that is positioned to encircle a nipple of one of the wearer's breasts, each central front panel opening having a circumference, and each central front panel opening being covered by a layered overlapping and interlocking central front panel opening cover;
wherein each central front panel opening cover includes an outer pair of flap members and an inner pair of flap members, the outer pair of flap members having a first outer fabric flap member and a second outer fabric flap member that extend from opposing sides of the central front panel opening so that an inner edge of the first outer fabric flap member overlaps an inner edge of the second outer fabric flap member along a first chord of the central front panel opening; the inner pair of flap members having a first inner fabric flap member and a second inner fabric flap member that extend from opposing sides of the central front panel opening so that an inner edge of the first inner fabric flap member overlaps an inner edge of the second inner fabric flap member along a second chord of the central front panel opening, provided that the first chord and the second chord are not parallel, so that each central front panel opening cover is configured such that by simultaneously separating the outer pair of flap members and separating the inner pair of flap members an access is provided to the nipple encircled by that central front panel opening;
a compartment formed between the inner fabric layer and the outer fabric layer of each front panel support zone, the compartment having a compartment perimeter where the inner fabric layer and the outer fabric layer are joined, so that the compartment extends at least partially around the circumference of the corresponding central front panel opening, and wherein each front panel defines an opening to the corresponding compartment that provides access to an interior of the compartment of that front panel;
two or more heating units, each heating unit configured to be inserted into the compartment of one of the front panels via the corresponding compartment opening and positioned within the compartment; each heating unit configured to be electrically coupled to a power source capable of energizing the heating unit while it is inserted into the compartment and thereby warming the wearer's breast where that heating unit is positioned;
a back portion configured to rest against a wearer's back and coupled to one of the pair of front panels on each side of the back portion;
a front fastener, disposed between the pair of front panels to fasten the pumping bra on the wearer; and
a pair of shoulder straps extending from the pair of front panels to the back portion, the shoulder straps having a racerback configuration to support the pumping bra on the wearer.

2. The pumping bra of claim 1, wherein each compartment is configured so that at least one heating unit can be variably positioned within the compartment in order to warm a desired portion of the wearer's breast.

3. The pumping bra of claim 1, wherein each compartment is configured so that the two or more heating units can be variably positioned within the compartment in order to desired portions of the wearer's breast.

4. The pumping bra of claim 1, wherein each compartment extends at least halfway around the circumference of its corresponding central front panel opening.

5. The pumping bra of claim 1, wherein the front fastener includes a zipper.

6. The pumping bra of claim 5, further comprising a fastener liner configured to prevent contact between the front fastener and the wearer.

7. The pumping bra of claim 1, wherein the first chord of the central front panel opening cover and the second chord of the central front panel opening cover are disposed at substantially a right angle to one another.

8. The pumping bra of claim 1, wherein the central front panel opening cover is configured so that by simultaneously separating the outer pair of flap members and separating the inner pair of flap members a portion of a breast pump can be inserted through the central front panel opening cover and be retained therein.

9. The pumping bra of claim 8, wherein the portion of the breast pump is a flange of a breast pump, and central front panel opening cover is configured so that the flange of the breast pump is secured by the central front panel opening cover.

10. The pumping bra of claim 9, wherein the flange of the breast pump is sufficiently secured by the central front panel opening cover that the breast pump can be operated hands-free.

11. The pumping bra of claim 1, wherein each heating unit is flexible enough to conform to the wearer's breast.

12. The pumping bra of claim 1, wherein each heating unit includes a substantially flat carbon fiber heating element.

13. The pumping bra of claim 1, wherein the power source includes an external power source.

14. The pumping bra of claim 1, wherein the power source includes a battery pack.

15. The pumping bra of claim 14, wherein the back portion further includes at least one external pocket configured to receive the battery pack.

16. The pumping bra of claim 1, wherein the power source includes a rechargeable battery pack and the two or more heating elements can be electrically coupled to the rechargeable battery pack via one or more powered USB ports.

17. The pumping bra of claim 1, further comprising a heating controller configured to permit an adjustment of a heat output of the two or more heating units, singly or in combination.

18. The pumping bra of claim 17, wherein the heating controller is configured to controllably provide 3-5 distinct heat outputs.
